(19) United States
(12) Patent Application Publication
(10) Pub. No.: US 2013/0233063 A1
(43) Pub. Date: Sep. 12, 2013

(54) Sensor for Determining Contact

(71) Applicants: Fancheng Wang, Beijing (CN); Peter Christopher Ellingson, Symmes, OH (US); Faiz Feisal Sherman, Mason, OH (US)

(72) Inventors: Fancheng Wang, Beijing (CN); Peter Christopher Ellingson, Symmes, OH (US); Faiz Feisal Sherman, Mason, OH (US)

(21) Appl. No.: 13/414,852

(22) Filed: Mar. 8, 2012

(51) Int. Cl. G01N 33/00

(52) U.S. Cl. 73/73

(57) ABSTRACT

A sensor for determining the level of contact between the consumer and the absorbent article and/or with bodily fluids is provided. The sensor comprises wetness sensels, pressure sensels, and combinations thereof. The sensor further comprises a microelectronic device and a power source. The sensor is attached to the microelectronic device providing a contiguous chamber with the sensor and the microelectronic device. The sensor may attach to a portion of skin.

### TECHNICAL FIELD

The present specification generally relates to an array of sensels that make up a sensor that allows one to detect and measure the contact and the distribution of fluid between skin and an absorbent article in real time and during any combination of body activities.

### BACKGROUND

Determining how a product interacts with a consumer and learning how the product functions in use can provide important information to the product's manufacturer or others. For example, the manufacturer may want to observe how different actions or activities by the consumer affect the orientation and functionality of the product. This is especially of interest in absorbent articles wherein the product performance and perception may be affected by its positioning and contact or lack of contact with the body. This may be of interest in order to identify product improvements as part of a product development program. Absorbent articles are in contact with sensitive skin and learning what areas have the highest level of contact during different activities can allow for product designs that improve overall comfort. Given that products and components thereof may exhibit different characteristics in a wet state versus a dry state, it may be also desirable to have a sensor that attaches to skin available to detect contact in terms of pressure and contact with bodily fluids between the skin and the absorbent article. Finally, it can be especially useful to have empirical data that correlates with a user's expressions of satisfaction or dissatisfaction with the article's performance under a variety of usage conditions.

### SUMMARY OF THE INVENTION

A sensor for detecting pressure and wetness having an array of sensels, a microelectronic device, and a power source. The sensels may be wetness sensels, pressure sensels, and combinations thereof. The sensor is attached to the microelectronic device providing a contiguous chamber with the sensor and the microelectronic device. The sensor detects pressure in the form of contact and contact with bodily fluids between an absorbent article and a portion of the body. The sensor may attach to a portion of skin.

### BRIEF DESCRIPTION OF THE DRAWINGS

The drawings are illustrative in nature and not intended to limit the subject matter defined by the claims. The following detailed description can be understood when read in conjunction with the following drawings, where like structure is indicated with like reference numerals and in which:

FIG. 1 schematically depicts a sensor according to one or more examples shown and described herein.

FIG. 2 schematically depicts a sensor according to one or more examples shown and described herein.

FIG. 3 represents the components of the sensor in a flow chart.

FIG. 4 schematically depicts an absorbent article according to one or more examples shown and described herein.

FIG. 5 schematically depicts an absorbent article according to one or more examples shown and described herein.

### DETAILED DESCRIPTION OF THE INVENTION

The present invention is directed to a sensor having an array of sensels for determining the level of contact between a user's body surfaces and an absorbent article and the transfer and distribution of bodily fluid between the consumer and the absorbent article. Using a plurality of sensors, one can observe and/or analyze the contact and interaction between the consumer and various absorbent articles. One can also observe and/or analyze the movement and distribution of fluid between the consumer and various absorbent articles. For example, it may be desirable to observe contact and the movement and distribution of fluid between the absorbent article and the consumer's skin while the consumer performs daily tasks including and not limited to walking, running, lying down, and transitioning from various positions to other positions in real time. Further, the sensor allows the manufacturer to determine potential areas of friction between the absorbent article and the consumers while possibly learning how quickly the fluid is absorbed and the duration of time it is in contact with the consumer. This information can allow the manufacturer to improve the design of the absorbent article to create an improved fit and experience for the consumer under dynamic, real-life usage conditions.

In a particularly useful mode, by concurrently mapping contact between the absorbent article and the user's skin in terms of both pressure and the location and duration of fluid before absorption, the empirical data afforded by the sensor can readily be compared and correlated with the user's comments regarding fit, effectiveness, and overall comfort of the article under real-world usage conditions. Importantly, such correlations can be used to establish a score relating to consumer confidence in the effectiveness of the article in securely imbibing and retaining bodily fluids.

The following sets forth a broad description of numerous different examples of the present disclosure. The description is to be construed as exemplary only and does not describe every possible combination of elements since describing every possible combination of elements would be impractical, if not impossible. It will be understood that any feature, characteristic, component, composition, ingredient, absorbent article, step or methodology described herein can be deleted, combined with or substituted for, in whole or part, any other feature, characteristic, component, composition, ingredient, absorbent article, step or methodology described herein. Numerous alternative combinations could be implemented, using either current technology or technology developed after the filing date of this patent, which would still fall within the scope of the claims.

As used herein, the term “comprising” means that the various components, ingredients, or steps, can be conjointly employed in practicing the present invention. Accordingly, the term “comprising” encompasses the more restrictive terms “consisting essentially of” and “consisting of.” Other terms may be defined as they are discussed in greater detail below.

As used herein, the term “computing device” refers to a system comprising an input device, a processor, at least one memory, and at least one output device coupled together via electrical circuitry or other suitable coupling means. Optionally, a network interface is also provided, for connecting a first computing device to a communications network.

Herein, the term “absorbent articles” refers to articles which absorb and contain body fluids or exudates, and more specifically, refers to articles which are placed against or in proximity to the body of the wearer to absorb and contain the various exudates discharged from the body. The term “disposable” is used herein to describe absorbent articles which are not intended to be laundered or otherwise restored or reused as an absorbent article (i.e., they are intended to be discarded after a single use, and preferably, to be disposed of in an environmentally compatible manner). Exemplary absorbent articles include disposable feminine hygiene absorbent articles. Such articles include tampons, sanitary napkins, interlabial products, incontinence devices, and pantiliners. Feminine hygiene articles do not include baby diapers.

As used herein, the term “memory” refers to a volatile or non-volatile storage media, such as ROM, SRAM, DRAM, and/or other types of RAM, flash memory, secure digital (SD) memory, registers, compact discs (CD), digital versatile discs (DVD), and/or other types of non-transitory computer-readable mediums. As is well known in the art, memory may include a plurality of distributed memory coupled via electrical circuitry.

As used herein, the term “output device” refers to a computer monitor, LCD display, flat-screen display (for example, gas-plasma, active matrix or LED display), or similar device (including a display of a personal digital assistant or other handheld or portable device) suitable for display of visual information, and/or a speaker or other suitable device for outputting audio information, as known in the art.

As used herein, the term “processor” relates to one or more suitable microprocessors well known in the art, which processes data and computing instructions received via input device or stored in memory. A processor executes computing instruction to perform processes such as those described below in accordance with the present invention. The processor may include any processing component operable to receive and execute instructions (such as from the data storage component and/or memory component). The input/output hardware may include and/or be configured to interface with a monitor, positioning system, keyboard, mouse, printer, image capturing device, microphone, speaker, and/or other device for receiving, sending, and/or presenting data.

As used herein, “interior” refers to any portion of a product or packaging that is not readily visible to the naked eye viewing the exterior of the product or packaging. In some examples, the interior of a product or packaging may include one or more of internal surfaces and internal components of the product or packaging. In some examples, the interior may either include or exclude liquids, semi-solids, solids, emulsions, and colloids introduced into the product or packaging that may or may not interact with the product or packaging, but do not form an integral part of the product or packaging.

Performance characteristics may be predicted (i.e., derived from a virtual model) or measured and/or derived from actual physical testing. Performance characteristics include any measurable parameter related to the overall performance of the absorbent article such as fluid handling, e.g. absorbency, fluid path, swelling of absorbent material, leakage, distribution of fluid, fluid storage and retention, characteristics of how fluid goes into an absorbent structure, interaction of materials with an absorbent core, wicking, kinetics of fluid movement (e.g., acquisition rate, spreading area). Performance characteristics also include fit of the article to a wearer. For example, performance characteristics pertaining to fit may include fit to the body, the behavior of absorbent material when dry/wet, fit of the product chassis on a body structure, e.g. mannequin or person, anatomical integration of the product with the body structure, comfort in use, and article behavior during movement.

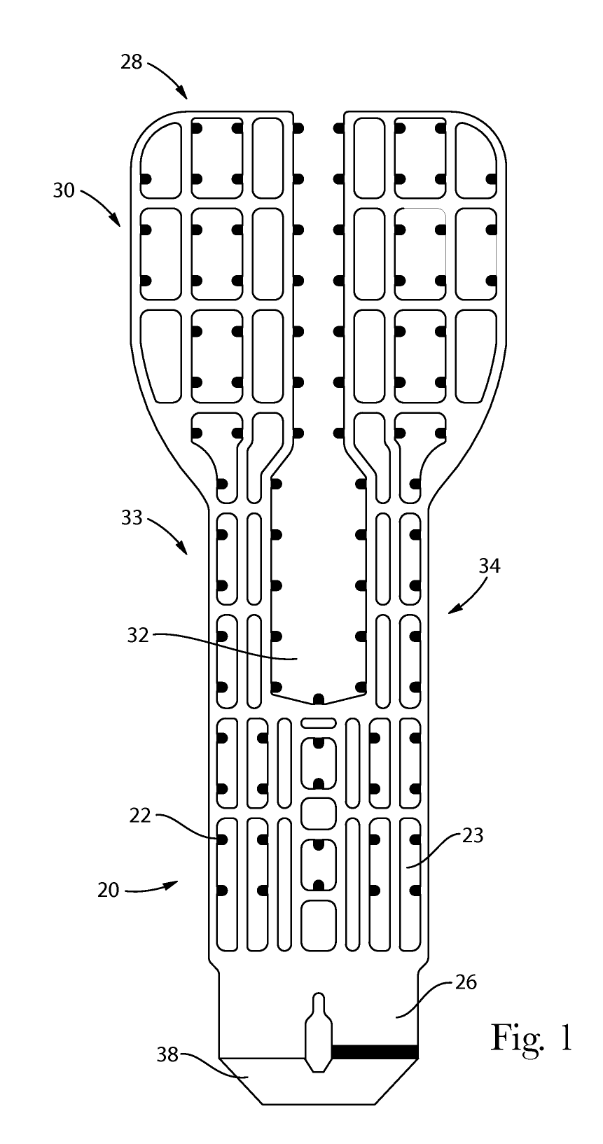

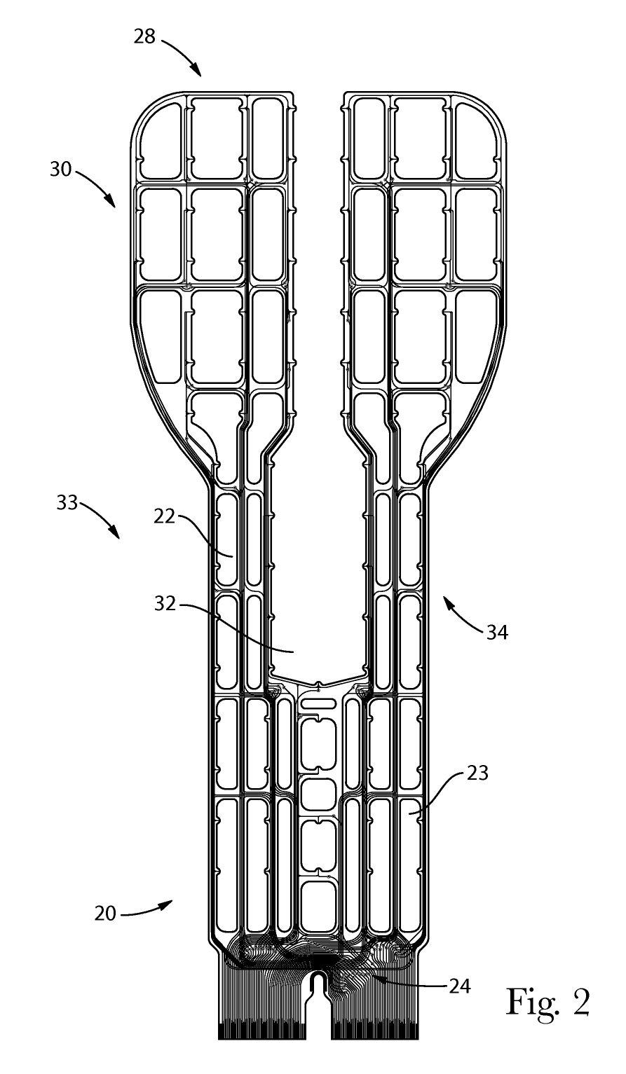

FIG. 1 and FIG. 2 depict a sensor 20 having wetness sensels 22, pressure sensels 23, and combinations thereof. The sensor 20 contains data acquisition hardware and data acquisition software. The sensor 20 may be made of an individual sensel 22, 23 or combinations of sensels 22, 23. The wetness sensels 22 and pressure sensels 23 may detect contact in the form of pressure and/or contact with bodily fluids.

The sensor 20 can be approximately about 0.1 mm to about 2 mm in thickness. The sensor 20 may be flexible thereby allowing it to fit the contours of the body.

The individual sensel 22 has a size that is preferably no greater than about 5 mm×5 mm, about 3 mm×3 mm, about 1 mm×1 mm, about 0.5 mm×0.5 mm, about 0.1 mm×0.1 mm, or even less. The distance between neighboring sensels is approximately about 5 mm to about 50 mm, about 5 mm to about 25 mm, about 10 mm to about 40 mm, and about 15 mm to about 30 mm. The overall length of the sensor 20 can be tailored for any length, for example, from about 50 mm to about 500 mm. The wetness sensels 22 and pressure sensels 23 can be arranged into rows 28 and columns 30. The rows 28 and columns 30 can be arranged separately or connected together to form grids. The width at one end of the sensor 20 is approximately about 20 mm to about 100 mm. Alternatively, the width may be as broad as 200 mm. The width on the other end of the sensor 20 is about 20 mm to about 200 mm. The central region 33 of the sensor 20 has a width of about 20 mm to about 100 mm, about 30 mm to about 80 mm, or about 40 mm to about 60 mm. Wetness sensels 22 and pressure sensels 23 may be distributed in the sensor 20 at one sensel 22, 23 for every one square centimeter, two sensels 22, 23 for every one square centimeter, and four sensels 22, 23 for every one square centimeter.

An optional open area 32 at the center of the sensor 20 may have a length of about 40 mm to about 80 mm and a width of about 10 mm to about 30 mm. The open area 32 at the center allows easy attachment to the body at the labial groove or groin region.

The sensor 20 may have wetness sensels 22, pressure sensels 23, or combinations thereof. The sensor 20 can measure pressure resulting from interaction between the absorbent article and the body. The registered pressure signals indicate contact between the absorbent article and the body. The sensor 20 may also detect contact with bodily fluids on an absorbent article or on the surface of the human or mannequin skin. The registered wetness signals indicate the presence, positioning, and uptake of bodily fluids by the absorbent article.

The sensor 20 registers data and can allow one to map pressure and contact between the absorbent article and the body in real time while also mapping the location of fluid and the duration that the fluid is in contact with the sensor 20. Using multiple sensors 20 may allow one to know what parts of the absorbent article are in contact with the body at different times and where and when fluid comes in contact with the sensor 20 during the use of the sensor 20.

The sensor 20 sends a signal when placed in contact with water or other water containing fluids. The signal may be converted to voltage or transmitted as a radio transmission. Using a plurality of sensors 20 may allow one to determine when and where the absorbent article comes in contact with the body during different activities. The sensor 20 may send a signal converted to voltage. The voltage may range from about 0.04V to about 0.5V. The signal may be converted by a signal amplifier and micro-processor prior to being recorded.

The sensor 20 can be electronically connected with the microelectronic device 26. The microelectronic device 26 may comprise a power source 38. The sensor 20 may include at least one radio-frequency identification tag and/or a power source 38 to power the electronic monitoring system. A user can place the sensor 20 adjacent to a body part that may discharge fluid on the skin of the body. The sensor 20 may record when it is placed in contact with the absorbent article or in contact with bodily fluids. This may allow one to determine how different activities affect the contact between an absorbent article and the wearer and, importantly, how the contact between the absorbent article and the wearer is affected by fluid being released by the body. When fluid is released from the body, fluid and/or the absorbent article may come in contact with the body allowing one or more sensors 20 to record contact with the absorbent article and/or the fluid. When contacted by an absorbent article or fluid, the sensor 20 transmits a signal to the microelectronic device 26.

The sensor 20 may transmit information pertaining to contact between the absorbent article and the skin and contact with bodily fluids. The sensor 20 may record up to at least about 8 hours, 16 hours, or about 24 hours of data that can then be transmitted. The sensor 20 may store up to one gigabyte of data for transfer to a system. The system includes a remote computing device, wherein the computing device is electronically connected with the microelectronic device 26 and the microelectronic device 26 transmits the information received from the sensor 20 to a computing device or system. The data may be transferred to the computing device via a USB connection, LAN connection, or alternate electronic relays including but not limited to Bluetooth™ or wireless technology, and/or smart fabric technology. The computing device can display or store the information, and a user can thereby review the information.

The computing device may run software like a data acquisition program that may be used to display, record, analyze, and/or organizing the information received from the sensor 20. The computing device can also include a storage means to store the information. The data acquisition program can control the scanning rate and adjust sensitivity to optimize the performance of the sensor 20. The data acquisition program may monitor data acquisition through a computer.

The sensor 20 may also include a transducer 24 or detector element. The transducer 24 or detector element is capable of detecting biological fluids and converting the signal resulting from the reaction into another signal which can be processed, measured, or quantified. The transducer 24 or detector element can use a physicochemical process, such as an optical, piezoelectric, or electrochemical method. To display the signal produced by the transducer 24 or detector element of the sensor 20 in a user friendly way, the sensor 20 array includes associated electronics or signal processors. The sensor 20 can utilize the aforementioned associated electronics used by the electronic monitoring system, including, but not limited to, smart fabric technology. The sensor 20 may also includes the associated electronics or signal processors to transfer the transducer 24 signal to the microelectronic device 26.

Various power sources may be used to power the components of the sensor 20. The power source 38 of the sensor 20 can include a battery, such as a rechargeable micro battery, a micro solar panel, by induction, or by a USB connection to a computer. The power supply may be sufficient to charge the sensor 20 for at least 8 hours, 16 hours, or for at least 24 hours.

Contact between the absorbent article and the body may be measured by the sensor 20 in various quantities. The sensor 20 may rate the contact on a 0-100 scale depending on the pressure exhibited on the sensor 20. The sensor scale may be divided by whole integers between 0 and 100. Alternatively, the sensor 20 may produce results in any known quantity or pressure measurement, such as, for example, in psi. The sensor 20 may also have a detection limit of 0.03 psi. The sensor 20 may have a pressure range of at least about 0.01 pounds per square inch to about 5 pounds per square inch.

The contact between the absorbent article and the body may affect product performance. At 100% contact between the absorbent article and the body, fluid may be acquired from the source and directly penetrate into pads. In contrast, at 0% contact between the absorbent article and the body, fluid freely moves around the exterior of the body without contacting the absorbent article. In addition, contact between the absorbent article and the body may also affect how consumers feel about the product. A consumer may deem that the product is not absorbing properly due to poor contact with the body. This may lead the consumer to experience a wet/drying feeling, a hot/sticky feeling, and/or a comfortable feeling.

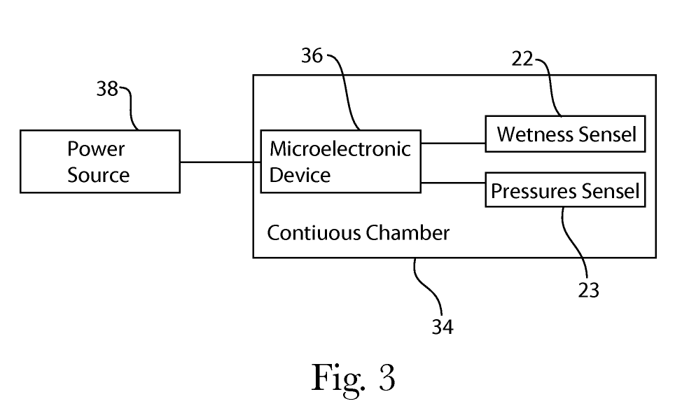

As shown in FIG. 3 and described above, the sensor has sensels for pressure 23 and wetness 22, a microelectronic device 26, and a power source 38. The sensor and the micro-electronic device make a contiguous chamber 34.

The sensor 20 can be made from a laminate flexible circuit material which contains alternate layers of film and pressure sensitive elements. The sensor 20 can also be made from a laminate flexible circuit material which contains alternate layers of lines of copper and Kapton film. These layers are cut to a desired geometry design and then bonded with heat and pressure. The end product can be a completely inert polyimide surface with small areas of exposed copper (sensels). All exposed copper is electroplated with a gold film, leaving virtually no exposed copper. The final assembly is coated with a hydrophobic coating, for example, a Krytox coating. Krytox is a fluoropolymer similar to liquid Teflon. The hydrophobic properties make it an excellent coating for removing liquid from the sensor 20.

One example of a laminate flexible circuit material which contains alternate layers of film and pressure sensitive elements is from Tekscan pressure sensors. These layers are cut to a desired geometry design and then bonded with heat and pressure or adhesive, the end product can be a completely inert surface, in which the sensor consists of two thin, flexible laminated polyester sheets (about 0.1 mm) which have electrically conductive electrodes deposited in varying patterns. In use, the sensor registers a pressure between the two surfaces. One such sensor is produced by Tekscan, Inc. located at 307 West First Street. South Boston, Mass. 02127-1309, USA.

The sensor 20 shape can be described as a “finger” type design. The design may consist of 2 to 20 columns 30 at one end. For example, the design may consist of 3, 4, 5, 6, 7, 8, 9, 10, 11, 12, 13, 14, 15, 16, 17, 18, 19, or 20 columns 30 at one end. The design may consist of 2 to 10 columns 30 in the middle with an empty area in between the columns 30 with multiple sensels 22, 23. For example, the design may consist of 3, 4, 5, 6, 7, 8, 9, or 10 columns 30 in the middle. The other end may consist of 2 to 20 columns 30. For example, the other end of the design may consist of 3, 4, 5, 6, 7, 8, 9, 10, 11, 12, 13, 14, 15, 16, 17, 18, 19, or 20 columns 30. Columns 30 of 2, 3, 4, 5, 6, 8, or 10 can be combined to a grid. Alternatively, the design may consist of equally spaced columns 30. Alternatively, the sensor 20 shape may consist of columns 30 that form a pattern where the columns 30 on one end are not aligned but are parallel to a column on the other end.

Sensors 20 are placed on the consumer in areas that may come in contact with the absorbent article. Such areas include the mons, leg crease, and buttocks area. The shape and dimensions of the sensor 20 allow for the sensor 20 to achieve a better body attachment. The shape and dimensions of the sensor 20 may also allow for the sensor 20 to achieve coverage and proper attachment despite the skin folding and area limitations that may be encountered with the female genital area.

Sensors 20 are placed on the body or skin of the person or mannequin at a minimum of one sensor 20 for every one square centimeter. Sensors 20 may be placed at one per several square centimeters or several sensors 20 for every one square centimeter. The distance between sensors 20 can be approximately about 5 mm to about 30 mm.

The sensors 20 may be placed using an adhesive. The sensors 20 may be placed using tape that does not interact with the function of the sensors 20. An example of tape that may be used is Tegaderm™ tape.

The sensor may 20 contain components and sensels 22, 23 purchased from PFC Flexible Circuits Limited located at 11 Canadian Road, Unit # 7, Scarborough, Ontario M1R 5G1, Canada and Quarter Century Design, LLC located at 1700 Thomas Paine Pkwy, Suite B, Centerville, Ohio 45459.

The sensor 20 may be sealed to be waterproof. The sensor 20 may have a satin material covering at least one side of the sensor 20. Alternatively, the covering may be made of any material that is considered suitable for textile or clothing including but not limited to cotton, rayon, and polyester.

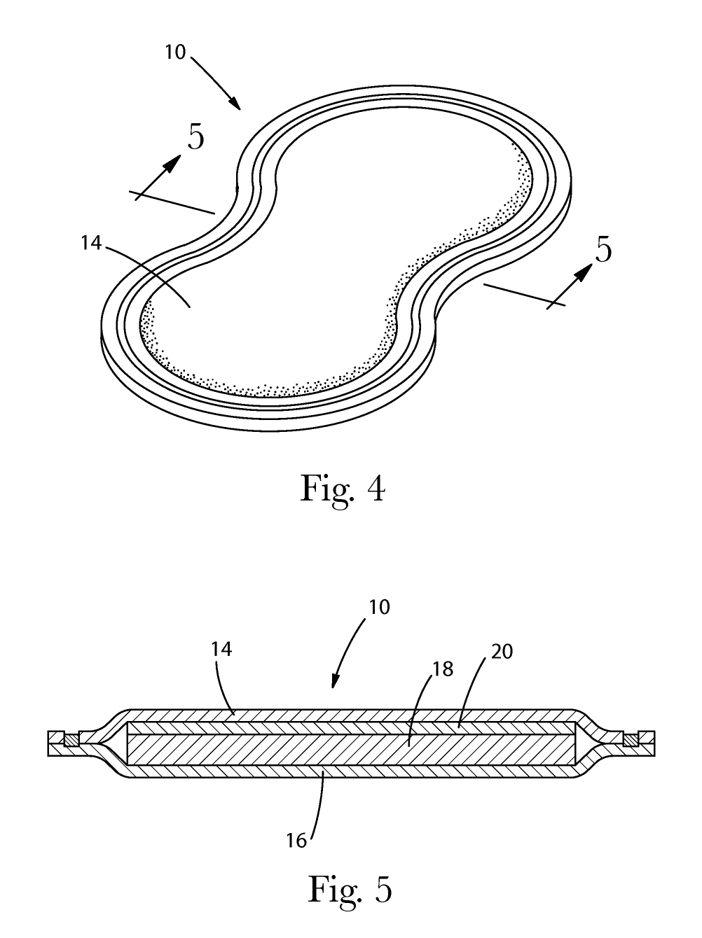

The invention can be used to determine contact with a variety of disposable absorbent articles, but is particularly useful in feminine hygiene products such as sanitary napkins and pantiliners. One embodiment of a disposable absorbent article that uses the invention is the sanitary napkin 10 shown in FIG. 4 and FIG. 5.

The illustrated sanitary napkin 10 has a body-facing upper side that contacts the user's body during use. The opposite, garment-facing lower side contacts the user's clothing during use. Suitable absorbent articles include any type of structures, from a single absorbent layer to more complex multi layer structures. Certain absorbent articles typically include a fluid pervious topsheet, a backsheet, which may be fluid impervious and/or may be water vapour and/or gas pervious, and an absorbent element often called “core” comprised there between.

A sanitary napkin 10 can have any shape known in the art for feminine hygiene articles, including the generally symmetric “hourglass” shape, as well as pear shapes, bicycle-seat shapes, trapezoidal shapes, wedge shapes or other shapes that have one end wider than the other. Sanitary napkins and pantyliners can also be provided with lateral extensions known in the art as “flaps” or “wings” (not shown in FIG. 4). Such extensions can serve a number of purposes, including, but not limited to, protecting the wearer's panties from soiling and keeping the sanitary napkin secured in place.

The upper side of a sanitary napkin generally has a liquid pervious topsheet 14. The lower side (seen in FIG. 5) generally has a liquid impervious backsheet 16 that is joined with the topsheet 14 at the edges of the product. An absorbent core 18 is positioned between the topsheet 14 and the backsheet 16. A secondary topsheet 20 may be provided at the top of the absorbent core 18, beneath the topsheet.

The topsheet 14, the backsheet 16, and the absorbent core 18 can be assembled in a variety of well-known configurations, including so called “tube” products or side flap products. Preferred sanitary napkin configurations are described generally in U.S. Pat. No. 4,950,264, “Thin, Flexible Sanitary Napkin” issued to Osborn on Aug. 21, 1990, U.S. Pat. No. 4,425,130, “Compound Sanitary Napkin” issued to DesMarais on Jan. 10, 1984; U.S. Pat. No. 4,321,924, “Bordered Disposable Absorbent Article” issued to Ahr on Mar. 30, 1982; U.S. Pat. No. 4,589,876, and “Shaped Sanitary Napkin With Flaps” issued to Van Tilburg on Aug. 18, 1987. Each of these patents is incorporated herein by reference.

The backsheet 16 and the topsheet 14 can be secured together in a variety of ways. Adhesives manufactured by H. B. Fuller Company of St. Paul, Minn. under the designation HL-1258 or H-2031 have been found to be satisfactory. Alternatively, the topsheet 14 and the backsheet 16 can be joined to each other by heat bonding, pressure bonding, ultrasonic bonding, dynamic mechanical bonding, or a crimp seal. A fluid impermeable crimp seal 24 can resist lateral migration (“wicking”) of fluid through the edges of the product, inhibiting side soiling of the wearer's undergarments.

The topsheet 14 may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be included of natural fibers (e.g., wood or cotton fibers), synthetic fibers (e.g., polymeric fibers such as polyester, polypropylene, or polyethylene fibers) or from a combination of natural and synthetic fibers. In one embodiment, the topsheet may be made of a hydrophobic material to isolate the wearer's skin from liquids which have passed through the topsheet 14. If the topsheet 14 is made of a hydrophobic material, at least the upper surface of the topsheet is treated to be hydrophilic so that liquids will transfer through the topsheet more rapidly. This diminishes the likelihood that body exudates will flow off the topsheet rather than being drawn through the topsheet and being absorbed by the absorbent core. In one embodiment, the topsheet 14 can be rendered hydrophilic by treating it with a surfactant. Suitable methods for treating the topsheet with a surfactant include spraying the topsheet material with the surfactant and immersing the material into the surfactant.

The topsheet 14 can include an apertured formed film. Apertured formed films can be used for the topsheet because they are pervious to body exudates and yet non-absorbent and have a reduced tendency to allow liquids to pass back through and rewet the wearer's skin. Thus, the surface of the formed film which is in contact with the body remains dry, thereby reducing body soiling and creating a more comfortable feel for the wearer. Suitable formed films are described in U.S. Pat. No. 3,929,135, entitled “Absorptive Structures Having Tapered Capillaries”, issued to Thompson on Dec. 30, 1975; U.S. Pat. No. 4,324,246 entitled “Disposable Absorbent Article Having A Stain Resistant Topsheet”, issued to Mullane, et al. on Apr. 13, 1982; U.S. Pat. No. 4,342,314 entitled “Resilient Plastic Web Exhibiting Fiber-Like Properties”, issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, issued to Ahr, et al. on Jul. 31, 1984; and U.S. Pat. No. 5,006,394 “Multilayer Polymeric Film” issued to Baird on Apr. 9, 1991.

The absorbent core 18 can be any absorbent member which is generally compressible, conformable, non-irritating to the wearer's skin, and capable of absorbing and retaining body fluids. The absorbent core may be manufactured in a wide variety of sizes and shapes (e.g., rectangular, hourglass, “T”-shaped, asymmetric, etc.) and from a wide variety of liquid-absorbent materials commonly used in disposable pull-on garments and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core 18 may vary (e.g., the absorbent core may have varying caliper zones, a hydrophilic gradient, a superabsorbent gradient, or lower average density and lower average basis weight acquisition zones; or may include one or more layers or structures). Further, the size and absorbent capacity of the absorbent core 18 may also be varied to accommodate wearers ranging from infants through adults. However, the total absorbent capacity of the absorbent core should be compatible with the design loading and the intended use of the absorbent article.

The absorbent core 18 may include other optional components. One such optional component is the core wrap, i.e., a material, typically but not always a nonwoven material, which either partially or totally surrounds the core. Suitable core wrap materials include, but are not limited to, cellulose, hydrophilically modified nonwoven materials, perforated films and combinations thereof.

The backsheet 16 can comprise a liquid impervious film. The backsheet 16 can be impervious to liquids (e.g., body fluids) and can be typically manufactured from a thin plastic film. However, typically the backsheet can permit vapours to escape from the disposable article. In an embodiment, a microporous polyethylene film can be used for the backsheet 16. A suitable microporous polyethylene film is manufactured by Mitsui Toatsu Chemicals, Inc., Nagoya, Japan and marketed in the trade as PG-P.

One suitable material for the backsheet 16 can be a liquid impervious thermoplastic film having a thickness of from about 0.012 mm (0.50 mil) to about 0.051 mm (2.0 mils), for example including polyethylene or polypropylene. Typically, the backsheet 16 can have a basis weight of from about 5 g/m² to about 35 g/m². However, it should be noted that other flexible liquid impervious materials may be used as the backsheet 16. Herein, “flexible” refers to materials which are compliant and which will readily conform to the general shape and contours of the wearer's body.

The backsheet 16 can be typically positioned adjacent the outer-facing surface of the absorbent core 18 and can be joined thereto by any suitable attachment device known in the art. For example, the backsheet may be secured to the absorbent core 18 by a uniform continuous layer of adhesive, a patterned layer of adhesive, or an array of separate lines, spirals, or spots of adhesive. Illustrative, but nonlimiting adhesives, include adhesives manufactured by H. B. Fuller Company of St. Paul, Minn., U.S.A., and marketed as HL-1358J. An example of a suitable attachment device including an open pattern network of filaments of adhesive is disclosed in U.S. Pat. No. 4,573,986 entitled “Disposable Waste-Containment Garment”, which issued to Minetola et al. on Mar. 4, 1986. Another suitable attachment device including several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Alternatively, the attachment device may include heat bonds, pressure bonds, ultrasonic bonds, dynamic mechanical bonds, or any other suitable attachment device or combinations of these attachment devices.

The backsheet 16 may be additionally secured to the topsheet 14 by any of the above-cited attachment devices.

The absorbent article 10 may also include such other suitable features as are known in the art including, but not limited to, re-closable fastening system, lotion, acquisition layers, distribution layers, wetness indicators, sensors, elasticized waist bands and other similar additional elastic elements and the like, belts and the like, waist cap features, containment and aesthetic characteristics and combinations thereof.

The afore-described contact and fluid data provided by using multiple sensors herein can be correlated with consumer perception data using typical scalar questionnaires. For example, the user can be asked to rate on a scale of one to ten such attributes as: initial comfort; initial fit; comfort after soiling; fit after soiling; comfort after re-soiling; fit after re-soiling; strike-through; overflow; and the like. An overall feeling of assurance that the article will—or—will-not fail in ordinary use can also be assessed, similarly.

Inasmuch as the foregoing analysis is largely a matter of subjective impression, the designer of absorbent articles is given very little detailed guidance for the design of improved products. However, by correlating such analysis with the empirical data afforded by the sensors herein, unexpected and heretofore design flaws and shortcomings can be identified with particularity, thereby assisting the overall product development effort.

For example, a panel of ten menstruating human female subjects may be assembled. Each subject may be provided with an absorbent article. The article is affixed to the subject's panties in the crotch region, in standard fashion associated with the use of such articles. The panties are donned by the subjects, which position the article in close contact with the vaginal area of each subject's body. A sensor 20 of the type shown in FIGS. 1 and 2 and containing both wetness sensels 22 and pressure sensels 23 is positioned between the article and the body of each subject in the vaginal area.

Each subject is instructed to undertake a variety of both active and passive tasks, including sitting, standing, stretching, bending forward from the wait, lying down (both prone and supine), walking, jogging, and crouching. For each task, the subjects are individually asked to evaluate the article for fit, comfort, and absorbency performance, including any instance of liquid strike-through or overflow, in the subject's panties. Evaluation is done on a scale of zero (unacceptable performance) to ten (excellent performance).

The subjects' subjective evaluations are correlated with the obtained pressure and wetness data afforded by the sensors. This allows the subjective data provided by the subjects to be translated into adjustments in the absorbency, geometry, and flexibility of the articles, corresponding to problem regions relating to wetness and pressure that have been identified by the sensors.

Although the present disclosure makes reference to particular examples, persons skilled in the art will recognize that changes may be made in form and detail without departing from the spirit and scope of the disclosure. All directional references (e.g., rear, front, left, right, top, bottom) are only used for identification purposes to aid the reader's understanding of the examples of the present invention, and do not create limitations, particularly as to the position, orientation, or use of the invention unless specifically set forth in the claims. It is intended that all matter contained in the above description or shown in the accompanying drawings shall be interpreted as illustrative only and not as limiting. Changes in detail or structure may be made without departing from the spirit of the disclosure as defined in the appended claims.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference, however the citation of any document is not construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular examples have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A sensor comprising:
a. An array of sensels;
b. a microelectronic device;
c. a power source;
wherein the sensor is attached to the microelectronic device providing a contiguous chamber with the sensor and the microelectronic device;
wherein the sensor may optionally adhere to a portion of skin; and
wherein the sensels comprise wetness sensels, pressure sensels, and combinations thereof.

2. The sensor of claim 1, wherein the sensor detects pressure in the range of 0.01 to about 5 pounds per square inch.

3. The sensor of claim 1, wherein the sensor measures a fluid path and fluid spreading on a body under various postures and activities.

4. The sensor of claim 1, wherein the sensor registers a signal for at least 24 hours.

5. The sensor of claim 1, wherein the sensor comprises an adhesive to adhere to skin.

6. The sensor of claim 1, wherein the sensor comprises at least 20 sensels.

7. The sensor of claim 6, wherein the sensor comprises four sensels for every square centimeter.

8. The sensor of claim 6, wherein the distance between neighboring sensels is between 5 mm to 25 mm

9. The sensor of claim 4, wherein the signal registered by the sensor is correlated with consumer perception data collected through questionnaires.

10. The sensor of claim 5, wherein the sensor comprises a geometric grid comprising sensels arranged in a set of rows and a set of columns.

11. The sensor of claim 10, wherein the set of rows of sensels and the set of columns of sensels are combined.

12. The sensor of claim 10, wherein the sensor has a center empty area with a dimension between about 10 to about 30 mm wide and between about 40 to about 80 mm long.

13. A sensor for detecting pressure or wetness, comprising:
a. a plurality of sensels;
b. a microelectronic device;
wherein the sensor is attached to the microelectronic device providing a contiguous chamber with the pressure sensor and the microelectronic device;
wherein the plurality of sensels detect pressure in the form of contact between an absorbent article and a portion of a body and contact with bodily fluids.

14. The sensor of claim 14, wherein the sensor detects pressure in the range of 0.01 to about 5 pounds per square inch.

15. The sensor of claim 14, wherein the microelectronic device comprises a power source.

16. The sensor of claim 16, wherein the sensor registers a signal for at least 24 hours.

17. The sensor of claim 14, wherein the sensor comprises an adhesive to adhere to skin.

18. The sensor of claim 14, wherein the sensor comprises of at least 20 sensels.

19. A method of observing the contact and interaction between a consumer and an absorbent article, the method comprising:
a. affixing an absorbent article to a consumer's panties in a crotch region;
b. donning the panties and positioning the article in close contact with the vaginal area;
c. adhering a plurality of sensors comprising an array of sensels to the area of the body located between the article and the body in the vaginal area, wherein the sensels detect contact in the form of pressure and/or contact with bodily fluids;
d. recording data during use while the consumer performs active and passive tasks; and
e. analyzing the recorded data to determine the contact and interaction between the consumer and the article.

20. The method of claim 19, wherein the method further comprises:
f. giving a questionnaire to the consumer; and
g. correlating sensor data with consumer perception data from the questionnaire.
